(19) United States
(12) Patent Application Publication
(10) Pub. No.: US 2008/0132896 A1
(43) Pub. Date: Jun. 5, 2008

(54) CURVED ORTHOPEDIC TOOL

(75) Inventors: William W. BOWEN, Willits, CA (US); Trung Ho Pham, Santa Rosa, CA (US); Heber Saravia, Santa Rosa, CA (US)

Correspondence Address: SHAY GLENN LLP, 2755 CAMPUS DRIVE, SUITE 210, SAN MATEO, CA, 94403, US

(73) Assignee: SONOMA ORTHOPEDIC PRODUCTS, INC., SANTA ROSA, CA (US)

(21) Appl. No.: 11/944,381

(22) Filed: Nov. 21, 2007

Related U.S. Application Data
(60) Provisional application No. 60/866,976, filed on Nov. 22, 2006.
(60) Provisional application No. 60/949,071, filed on Jul. 11, 2007.

(51) Int. Cl. A61B 17/58; A61B 17/32

(52) U.S. Cl. 606/80; 606/167

(57) ABSTRACT

A method of forming an opening in a bone comprising: inserting a tip of a curved rigid tool into an entry point in the bone and advancing the tool along a curved path into the bone. Another aspect of the invention provides an orthopedic tool comprising a rigid curved body having a diameter suitable for insertion into a bone and a sharp tip at a distal end of the curved body.

### CROSS REFERENCE TO RELATED APPLICATIONS

This application claims the benefit under 35 U.S.C. § 119 of the following U.S. provisional applications, the disclosures of which are incorporated herein by reference: U.S. Ser. No. 60/866,976, “SURGICAL TOOLS FOR USE IN DEPLOYING BONE REPAIR DEVICES,” filed Nov. 22, 2006; and U.S. Ser. No. 60/949,071, “FRACTURE FIXATION DEVICE, TOOLS AND METHODS,” filed Jul. 11, 2007.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

Many orthopedic procedures require the formation of an opening in a bone to accommodate the insertion of an implant or tool. For example, sharp tipped trocars have been used to create an opening in a fractured long bone for the purpose of implanting an intramedullary nail to repair the fracture. The opening made by the trocar may be subsequently enlarged with a reamer. Such trocars typically have a straight body leading to a sharp tip in line with the straight body.

### SUMMARY OF THE INVENTION

Some new orthopedic procedures may require or at least benefit from a curved opening in the target bone. For example, during the fracture repair procedure described in US 2006/0264950, access to the intramedullary space is made along a curved path through an entry point on a bony protuberance of the fractured bone. This invention provides an orthopedic tool that will help form this curved opening in the target bone.

One aspect of the invention provides a method of forming an opening in a bone including the steps of inserting a tip of a curved rigid tool into an entry point in the bone and advancing the curved tool along a curved path into the bone. In some embodiments, prior to the step of inserting the tip, the method includes the step of inserting a guidewire through the entry point into the bone, the method further comprising advancing the curved tool over the guidewire.

In embodiments in which the bone is a long bone, the entry point may be a bony protuberance of the long bone. In some embodiments, the advancing step includes the step of compressing internal bone tissue displaced by the advancing tool. In embodiments of the invention, the curved rigid tool may have an S-shaped body portion and/or a C-shaped body portion.

Another aspect of the invention provides an orthopedic tool with a rigid curved body having a diameter suitable for insertion into a bone and a sharp tip at a distal end of the curved body. In some embodiments, the curved body has a diameter between 0.125 and 0.5 inches and a radius of curvature between 0.5 inches and 6 inches.

In some embodiments, the body has an S-shaped portion and/or a C-shaped portion. Some embodiments provide a guidewire lumen extending through the body and tip of the tool. There may also be an integral handle. The tip may be bicuspid shaped, tricuspid shaped, fluted as a reamer or conically shaped.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a cross-sectional view of a fractured bone.

FIG. 2 is a cross-sectional view of the fractured bone of FIG. 1 with a curved opening formed therein.

FIG. 3 is a cross-sectional view of the fractured bone of FIGS. 1 and 2 with a fracture repair device implanted therein.

FIG. 4 is a perspective view of an orthopedic tool according to one embodiment of the invention.

FIG. 5 is a perspective view of an orthopedic tool according to another embodiment of the invention.

FIG. 6 is a cross-sectional view of a body portion of the tool of FIG. 5.

FIG. 7 is a perspective view of an orthopedic tool according to another embodiment of the invention.

FIG. 8 is a cross-sectional view of a body portion of the tool of FIG. 7.

### DETAILED DESCRIPTION OF THE INVENTION

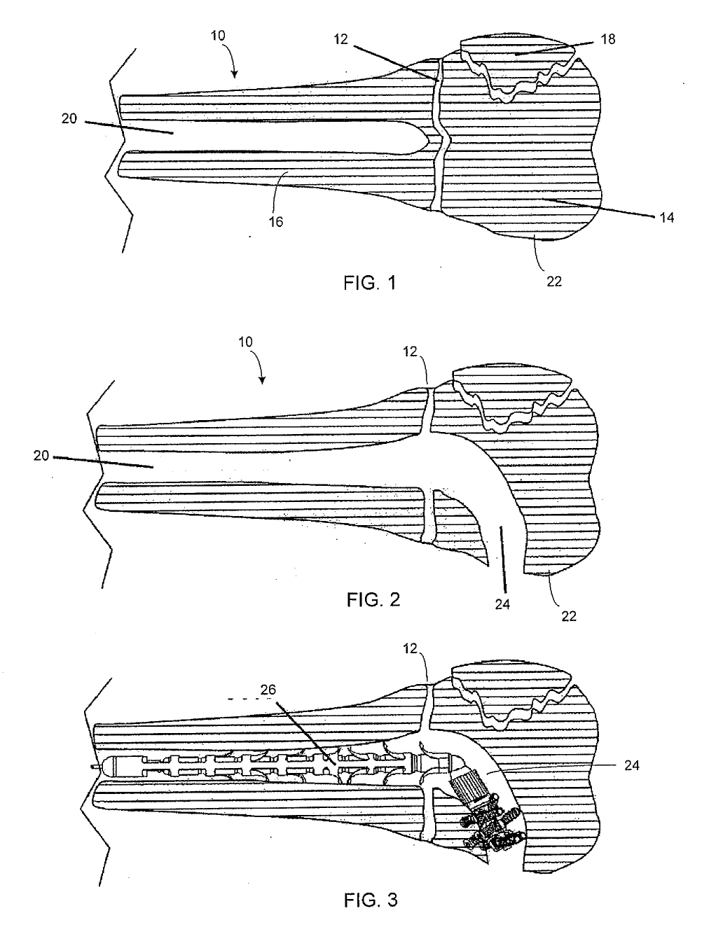

FIG. 1 shows a fractured long bone 10, such as a radius. A transverse fracture line 12 lies between the metaphysis 14 and the diaphysis 16. The metaphysis also has a bone chip 18.

In order to repair this fracture with an implanted device, access to the bone's intramedullary space 20 must be provided. In this embodiment, the target access site is a bony protuberance 22 in the metaphysis. As shown in FIG. 2, a curved channel, access port or opening 24 is formed at the target access site and extends into the intramedullary canal. A bone fracture repair device 26 can then be delivered to and deployed in opening 24 to repair the fracture, as shown in FIG. 3. Further details of bone fracture repair devices suitable for being delivered and deployed through such curved openings can be found in US 2006/0264950 and in a concurrently filed US patent application titled, “FRACTURE FIXATION DEVICE, TOOLS AND METHODS” naming Heber Saravia as a first inventor and having attorney docket number 10176-703.201.

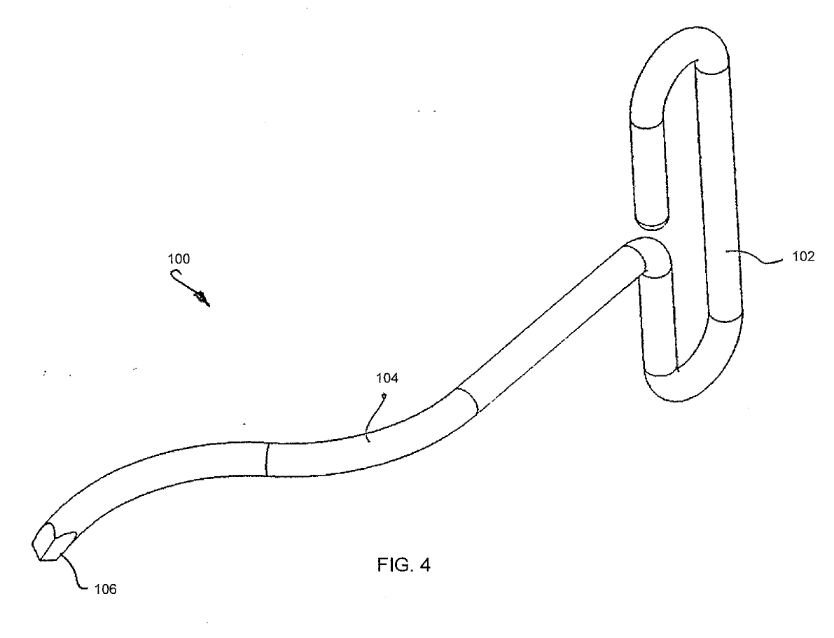

FIG. 4 shows one embodiment of an orthopedic tool suitable for making a curved opening or curved access port in a bone, such as the curved opening shown in FIGS. 2-3. Curved trocar 100 has a integral handle 102 leading to a curved body 104. Body 104 may be S-shaped as shown or C-shaped. The curvature of body 104 substantially matches the desired curvature of the opening for the bone fixation device, such as that shown in FIGS. 2-3. In some embodiments, the body has a radius of curvature between 0.5 inches and 6 inches, depending on the application. Body 104 ends in a sharp tip 106. Tip 106 may be bicuspid shaped as shown or, alternatively, tricuspid or conical.

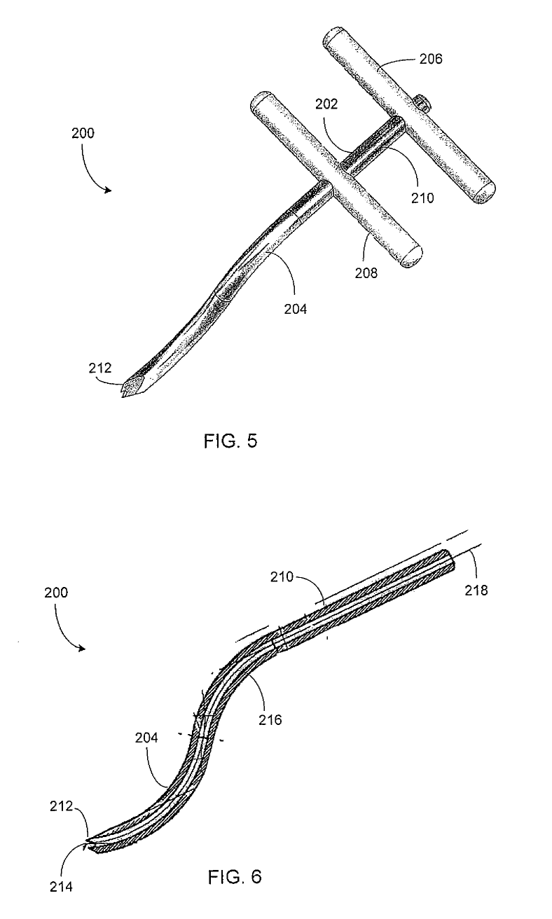

FIGS. 5 and 6 show another embodiment of an orthopedic tool according to the invention. As in the FIG. 4 embodiment, curved trocar 200 has an integral handle 202 leading to a curved body 204. Body 204 may be S-shaped as shown or C-shaped, and the curvature of body 204 substantially matches the desired curvature of the opening for the bone fixation device, such as that shown in FIGS. 2-3. In some embodiments, the body has a radius of curvature between 0.5 inches and 6 inches and a diameter between about 0.125 and 0.500 inches, depending on the application. A handle extension 210 connects two handle cross-pieces 206 and 208 to body 204.

Body 204 ends in a sharp tip 212. Tip 212 may be tricuspid as shown or, alternatively, bicuspid or conical. A guidewire opening 214 formed in tip 212 leads to a guidewire lumen 216 extending through body 204 and handle extension 210. FIG. 6 shows a guidewire 218 in place within lumen 216. (Cross-pieces 206 and 208 are omitted from FIG. 6 for clarity.)

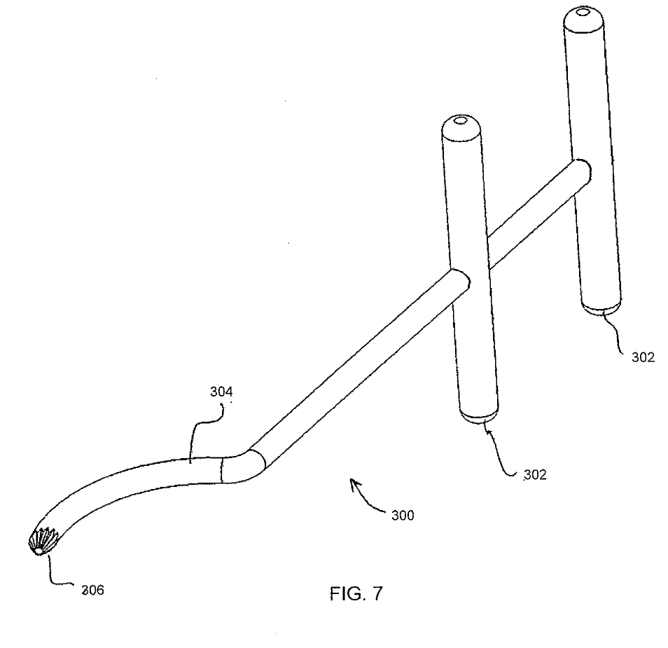

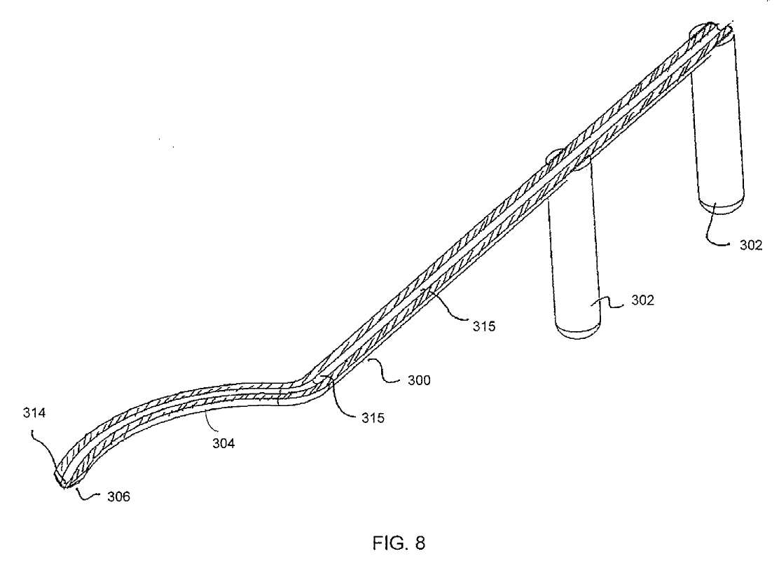

FIGS. 7 and 8 show another embodiment of an orthopedic tool according to the invention. As in the FIG. 5 embodiment, curved trocar 300 has an integral handle 302 leading to a curved body 304. Body 304 may be C-shaped as shown or S-shaped, and the curvature of body 304 substantially matches the desired curvature of the opening for the bone fixation device such as shown in FIGS. 2-3. Tip 306 may be fluted as shown, or, alternatively, bicuspid, tricuspid or conical. A guidewire opening 314 formed in tip 306 leads to a guidewire lumen 315 extending through body 304 and handle extension 302.

In use to repair a bone fracture, a target entry point of a bone (such as at a bony protuberance) may be exposed by an incision, and the sharp tip of the curved trocar 100 or 200 may be pressed through the surface of the bone to form a curved opening or channel in the bone. If a guidewire is used for the procedure, the guidewire may be inserted first into the bone at the target entry point, and the curved trocar 200 may be advanced over the guidewire to form the curved opening. The opening may optionally be widened using a reamer, drill or a larger-size curved trocar or other suitable device. Thereafter, a bone fracture repair device may be inserted into the curved opening to repair the fracture.

Other orthopedic procedures may also be performed using the devices and methods of this invention.

What is claimed is:

1. A method of forming an opening in a bone comprising:
inserting a tip of a curved rigid tool into an entry point in the bone; and
advancing the tool along a curved path into the bone.

2. The method of claim 1 further comprising, prior to the step of inserting the tip, inserting a guidewire through the entry point into the bone, the method further comprising advancing the tool over the guidewire.

3. The method of claim 1 wherein the bone comprises a long bone, the entry point comprising a bony protuberance of the long bone.

4. The method of claim 1 wherein the advancing step comprises compressing bone tissue.

5. The method of claim 1 wherein the curved rigid tool comprises an S-shaped body portion.

6. The method of claim 1 wherein the curved rigid tool comprises a C-shaped body portion.

7. An orthopedic tool comprising: a rigid curved body having a diameter suitable for insertion into a bone and a sharp tip at a distal end of the curved body.

8. The orthopedic tool of claim 7 wherein the body has a curved portion having a radius of curvature between 6 inches and 0.5 inches.

9. The orthopedic tool of claim 7 wherein the body comprises an S-shaped portion.

10. The orthopedic tool of claim 7 wherein the body comprises a C-shaped portion.

11. The orthopedic tool of claim 7 further comprising a guidewire lumen extending through the body and tip.

12. The orthopedic tool of claim 7 further comprising an integral handle.

13. The orthopedic tool of claim 7 wherein the tip is bicuspid shaped.

14. The orthopedic tool of claim 7 wherein the tip is tricuspid shaped.

15. The orthopedic tool of claim 7 wherein the tip is conically shaped

16. The orthopedic tool of claim 7 wherein the tip is fluted.
